(19) United States
(12) Patent Application Publication
(10) Pub. No.: US 2019/0142278 A1
(43) Pub. Date: May 16, 2019

(54) INFORMATION PROCESSING APPARATUS, INFORMATION PROCESSING METHOD, AND PROGRAM

(71) Applicant: CANON KABUSHIKI KAISHA, Tokyo (JP)

(72) Inventors: Shoya Sasaki, Yokohama-shi (JP); Kazuhito Oka, Tokyo (JP); Kenichi Nagae, Yokohama-shi (JP)

(21) Appl. No.: 16/189,759

(22) Filed: Nov. 13, 2018

(30) Foreign Application Priority Data
Nov. 15, 2017 (JP) 2017-220335

(51) Int. Cl. A61B 5/00; G06T 7/70; G01N 29/06; G01N 29/24

(52) U.S. Cl. CPC A61B 5/0095; G01N 29/2418; G01N 29/0654; G06T 7/70

(57) ABSTRACT

An information processing apparatus according to the present disclosure includes an incidental information obtaining unit configured to obtain incidental information of a plurality of photoacoustic image data pieces designated based on a user's instruction in a photoacoustic image data set, a type information obtaining unit configured to obtain type information indicating a type of composition image data, and an adaptability acquiring unit configured to acquire, based on the incidental information, an adaptability between a combination of the plurality of photoacoustic image data pieces and computation of the composition image data of the type indicated by the type information.

### BACKGROUND

### Field of the Disclosure

The present disclosure relates to an information processing apparatus, an information processing method, and a program for performing processing relating to photoacoustic image data.

### Description of the Related Art

Photoacoustic imaging has been known to apply pulsed light to an object such as a living body and displays a photoacoustic image indicating information within the object based on acoustic waves (hereinafter, called photoacoustic waves) because of a photoacoustic effect.

Such photoacoustic imaging can generate photoacoustic image data representing a space distribution of a sound pressure (initial sound pressure) and optical absorption coefficients of acoustic waves generated by optical absorption.

In the photoacoustic imaging, a plurality of photoacoustic image data pieces acquired by a photoacoustic apparatus can be used to generate a new image data piece.

Japanese Patent Laid-Open No. 2017-35407 discloses that a plurality of light beams having different wavelengths are irradiated to acquire an absorption coefficient distributions corresponding to the wavelengths. Japanese Patent Laid-Open No. 2017-35407 discloses that information regarding an oxygen saturation of an object is computed by using a plurality of absorption coefficient distributions corresponding to a plurality of wavelengths.

### SUMMARY

An information processing apparatus according to the present disclosure includes an incidental information obtaining unit configured to obtain incidental information of a plurality of photoacoustic image data pieces designated based on a user's instruction in a photoacoustic image data set, a type information obtaining unit configured to obtain type information indicating a type of composition image data, and an adaptability acquiring unit configured to acquire, based on the incidental information, an adaptability between a combination of the plurality of photoacoustic image data pieces and computation of the composition image data of the type indicated by the type information.

Further features of the present invention will become apparent from the following description of exemplary embodiments with reference to the attached drawings.

### BRIEF DESCRIPTION OF THE DRAWINGS

FIG. 1 is a block diagram illustrating a system according to an embodiment of the present disclosure.

FIG. 2 is a flow chart illustrating a method for computing composition image data according to an embodiment of the present disclosure.

FIG. 3 illustrates a graphical user interface (GUI) for designating composition image data according to an embodiment of the present disclosure.

FIG. 4 illustrates a GUI displaying combinations of photoacoustic image data pieces according to an embodiment of the present invention.

FIG. 5 illustrates a GUI displaying candidates for an adaptable photoacoustic image data according to an embodiment of the present disclosure.

FIG. 6 illustrates a GUI in a case where inadaptable photoacoustic image data piece is designated according to an embodiment of the present disclosure.

FIG. 7 illustrates a GUI displaying an image based on a composition image data according to an embodiment of the present disclosure.

FIG. 8 is a detail block diagram illustrating a photoacoustic apparatus and an information processing apparatus according to an embodiment of the present invention.

FIG. 9 is a schematic diagram illustrating a probe according to an embodiment of the present disclosure.

FIG. 10 is a block diagram illustrating a computer and its peripheral configuration according to an embodiment of the present disclosure.

FIG. 11 is a flow chart in a photoacoustic image data generating method according to an embodiment of the present disclosure.

### DESCRIPTION OF THE EMBODIMENTS

With reference to drawings, embodiments of the present disclosure will be described below. However, the dimensions, qualities, shapes and relative arrangements thereof in the configurations which will be described below, should be changed in accordance with the configuration and conditions of apparatuses to which the present disclosure is to be applied. It is not intended that the scope of the present disclosure is limited by the following descriptions.

Photoacoustic image data acquired by a system according to the present disclosure reflect an absorbing quantity and an absorption ratio of light energy. Photoacoustic image data are image data representing a space distribution of object information that is at least one of a generated sound pressure (initial sound pressure), an optical absorption energy density, and am optical absorption coefficient of photoacoustic waves. The photoacoustic image data may be image data representing a two-dimensional space distribution and image data representing a three-dimensional space distribution. The system according to the present disclosure can compute composition image data of an object by using a plurality of photoacoustic image data pieces. The composition image data is image data computed from a plurality of photoacoustic image data pieces. Typically, the composition image data is information indicative of a function of an object and will also be called functional information. For example, the composition image data may be a glucose concentration, a collagen concentration, a melanin concentration, volume fractions of fat and water, and other concentration information of substances contained in an object. The composition image data may be difference information among a plurality of photoacoustic image data pieces by which a change over time of a state of an object can be identified.

For computing composition image data of an object by using a plurality of photoacoustic image data pieces, a user may designate photoacoustic image data pieces not adaptable for computing of desired composition image data, which may not result in a desired adaptable composition image data. Accordingly, the present disclosure provides an information processing apparatus which may facilitate designation of a photoacoustic image data piece adaptable for computing of desired composition image data.

A configuration of a system and an information processing method according to an embodiment will be described below.

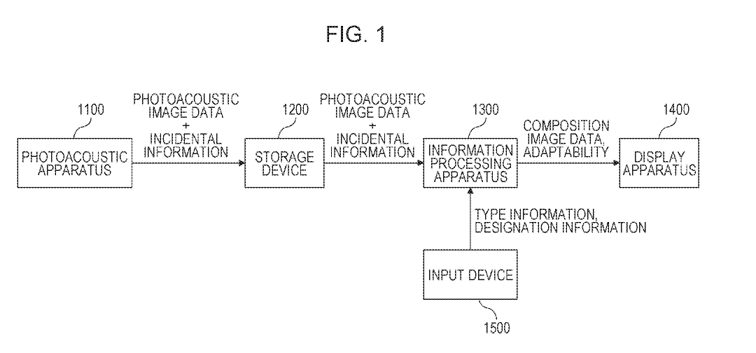

With reference to FIG. 1, a system according to this embodiment will be described. FIG. 1 is a block diagram illustrating a configuration of a system according to this embodiment. The system according to this embodiment includes a photoacoustic apparatus 1100, a storage device 1200, an information processing apparatus 1300, a display apparatus 1400, and an input device 1500. Here, data can be transmitted and be received between apparatuses and devices in a wired or wireless manner.

The photoacoustic apparatus 1100 is configured to photograph an object to generate photoacoustic image data and outputs it to the storage device 1200. The photoacoustic apparatus 1100 is an apparatus which generates information on a characteristic value at a plurality of positions within an object by using a reception signal acquired by receiving photoacoustic waves generated from irradiated light. In other words, the photoacoustic apparatus 1100 is an apparatus which generates a space distribution of characteristic value information originated from photoacoustic waves as medical image data (photoacoustic image data).

Photoacoustic image data generated by the photoacoustic apparatus 1100 reflects an absorbing quantity and absorption ratio of light energy. The photoacoustic image data generated by the photoacoustic apparatus 1100 may be information regarding a sound pressure (initial sound pressure) of an occurring acoustic wave, a light energy absorption density, light absorption coefficient, or a concentration of a substance contained in tissue, for example. A concentration of a substance may refer to an oxygen saturation, a total hemoglobin concentration, an oxyhemoglobin or a deoxyhemoglobin concentration, for example. The information regarding a concentration of a substance may be a glucose concentration, a collagen concentration, a melanin concentration, volume fractions of fat and water.

The storage device 1200 may be a storage medium such as a ROM (Read only memory), a magnetic disk, or a flash memory. The storage device 1200 may be a storage server over a PACS (Picture Archiving and Communication System) network.

The information processing apparatus 1300 is an apparatus configured to process information such as photoacoustic image data and incidental information of the photoacoustic image data stored in the storage device 1200.

Units responsible for a computing function of the information processing apparatus 1300 can include a processor such as a CPU (central processing unit), a GPU (graphics processing unit) and a computing circuit such as an FPGA (Field Programmable Gate Array) chip. These units may include a single processor and a computing circuit but, alternatively, may include a plurality of processors and a computing circuit.

A unit responsible for a storage function of the information processing apparatus 1300 may be a non-transitory storage medium such as a ROM (Read only memory), a magnetic disk or a flash memory. The unit responsible for the storage function may be a volatile medium such as a RAM (random access memory). It should be noted that a storage medium configured to store a program is a non-transitory storage medium. The unit responsible for the storage function may include a plurality of storage medium instead of one storage medium.

The unit responsible for the control function of the information processing apparatus 1300 may be an computing operation element such as a CPU. The unit responsible for a control function controls actions of components of the system. The unit responsible for the control function may control components of the system in response to an instruction signal for an operation such as a measurement start from an input unit. The unit responsible for the control function may read out program code stored in a storage unit and control operations of the components of the system.

The display apparatus 1400 is a display such as a liquid crystal display or an organic electroluminescence (EL). The display apparatus 1400 may display GUIs for operating an image or an apparatus.

The input device 1500 may be an operating console including a mouse and a keyboard which can be operated by a user. The display apparatus 1400 may include a touch panel and the display apparatus 1400 may be used as the input device 1500.

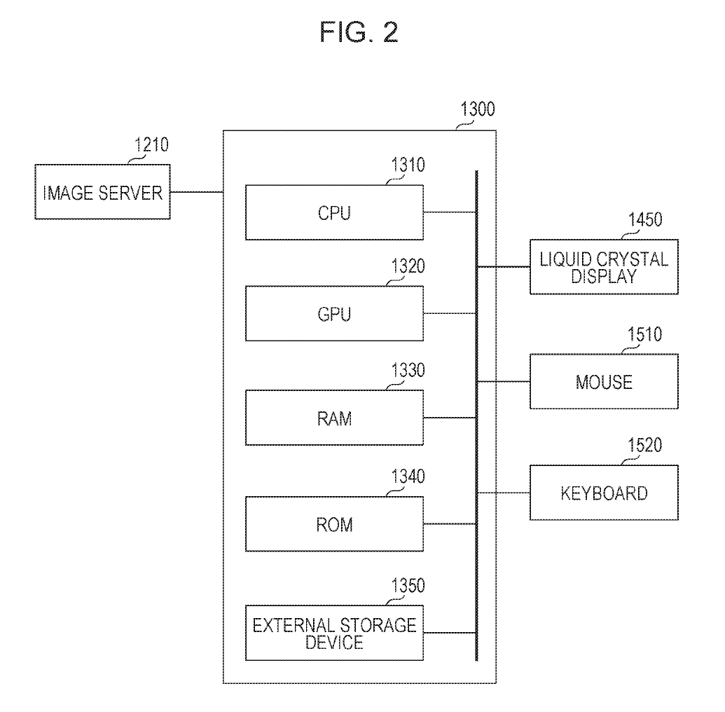

FIG. 2 illustrates an specific example of a configuration of the information processing apparatus 1300 according to this embodiment. The information processing apparatus 1300 according to this embodiment includes a CPU 1310, a GPU 1320, a RAM 1330, a ROM 1340, and an external memory 1350. To the information processing apparatus 1300, a liquid crystal display 1450 as the display apparatus 1400 and the mouse 1510 and keyboard 1520 as the input device 1500 are connected. Furthermore, the information processing apparatus 1300 is connected to the image server 1210 as the storage device 1200 such as a PACS (picture archiving and Communication system). Thus, image data can be stored on the image server 1210, and image data on the image server 1210 can be displayed on the display apparatus 1400.

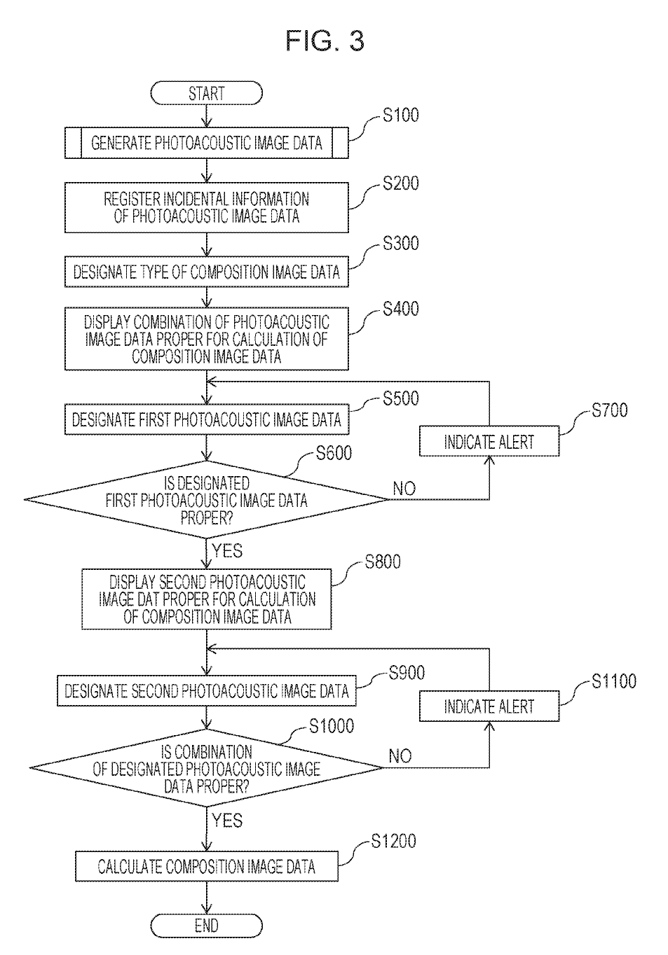

FIG. 3 illustrates a flow for acquiring composition image data by using the system according to this embodiment. Hereinafter, the flow for acquiring composition image data according to this embodiment will be described with reference to FIG. 3.

### S100: Processing for Generating Photoacoustic Image Data

The photoacoustic apparatus 1100 generates photoacoustic image data by photographing an object and outputs the photoacoustic image data to the storage device 1200. Details of the method for generating a photoacoustic image data will be described below.

### S200: Processing for Registering Incidental Information of Photoacoustic Image Data

The photoacoustic apparatus 1100 registers the incidental information in association with the photoacoustic image data and causes the storage device 1200 to store the photoacoustic image data. The storage device 1200 may store a photoacoustic image data piece generated by one photographing operation as well as a photoacoustic image data set in association with incidental information. The photoacoustic image data set, which will be described below, may be whole image data stored in the storage device 1200 or partial image data in the storage device 1200. A user may use the input device 1500 to change the incidental information of photoacoustic image data stored in the storage device 1200.

The incidental information may be information regarding patient information or photoacoustic image data. The patient information may include, for example, at least one information piece such as patient's ID, name, birthday, sex, past examination date and time, a photographed region and a photographing modality. Information regarding photoacoustic image data may include at least one information piece of for example, a photographed date and time, a photographed region, a measured wavelength, an initial sound pressure distribution, an optical absorption coefficient distribution, and a type (image type) of photoacoustic image data.

### S300: Processing for Designating a Type of Composition Image Data

A user may use the input device 1500 to designate a type of composition image data to compute. The information processing apparatus 1300 as a type information obtaining unit is configured to obtain type information indicating a type of composition image data to compute by the user through the input device 1500. According to this embodiment, the term “type information” can refer to request information defining a type to compute by a user. For example, a user can designate a desired type of composition image data from a list of a plurality of types of composition image data displayed on the display apparatus 1400. Any method may be applied for designating a desired type of composition image data from a plurality of types of composition image data. It should be noted that the type information may be information defining a predetermined type of composition image data.

Here, when a user uses the input device 1500 to designate patient information (such as a patient ID), the computer 150 may be caused to distinguishably display on the display apparatus 1400 a type of composition image data which can be computed from a combination of photoacoustic image data pieces corresponding to the designated patient. The computer 150 may differentiate the display mode for an item representing composition image data which can be computed from a combination of the photoacoustic image data pieces corresponding to the designated patient information and the display mode for an item representing another composition image data. The display mode for an item representing composition image data which can be computed and the display mode for photoacoustic image data pieces to be used for the computing may be displayed in association.

In this case, the information processing apparatus 1300 obtains patient information designated by the user through the input device 1500. The information processing apparatus 1300 obtains the incidental information of a photoacoustic image data set stored in the storage device 1200. The information processing apparatus 1300 determines photoacoustic image data corresponding to the patient information with reference to the incidental information of the photoacoustic image data set stored in the storage device 1200. For example, the information processing apparatus 1300 determines photoacoustic image data pieces including an identical patient ID as incidental information from the photoacoustic image data set with reference to the patient IDs associated with the photoacoustic image data set.

The information processing apparatus 1300 further computes information indicating composition image data which can be computed from a combination of photoacoustic image data pieces corresponding to patient information and transmits the information to the display apparatus 1400. The information processing apparatus 1300 computes composition image data which can be computed with reference to an image type of photoacoustic image data pieces corresponding to patient information as incidental information, wavelengths used for the photographing, and photographed dates and times. For determination of a composition image data that can be computed, for example, a range of wavelengths applicable for each composition image data, an interval equal to or longer than or equal to or shorter than a predetermined time period of photographing dates and times, or a region to be photographed may be determined separately, and these ranges may be changed by a user.

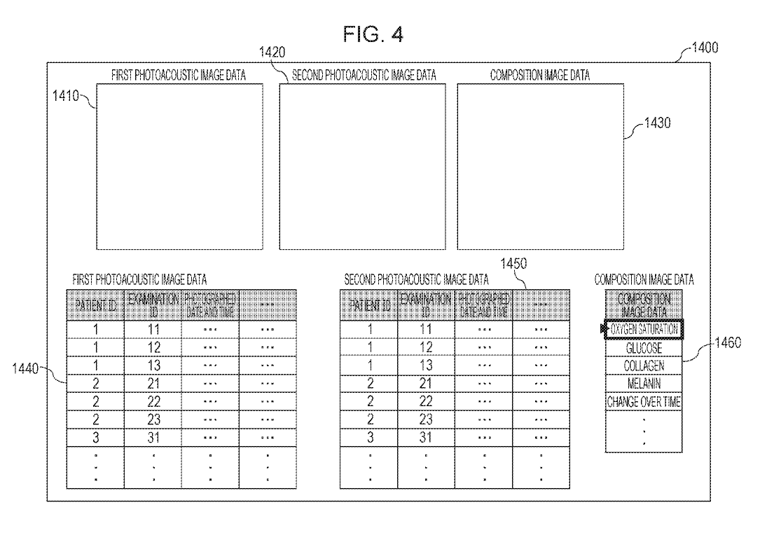

FIG. 4 to FIG. 8 are GUIs (graphical user-interfaces) to be displayed on the display apparatus 1400 according to this embodiment. A display region 1410 displays an image based on a first photoacoustic image data piece, which will be described below. A display region 1420 displays an image based on a second photoacoustic image data piece, which will be described below. A display region 1430 displays an image based on composition image data. A list 1440 is a list of candidates for the first photoacoustic image data piece to be used for computing composition image data. A list 1450 is a list of candidates for the second photoacoustic image data piece to be used for computing the composition image data. The list 1440 and the list 1450 have a plurality of items indicative of candidates for the respective photoacoustic image data pieces. The items corresponding to the photoacoustic image data set displayed on the list 1440 and list 1450 correspond to photoacoustic image data set stored in the storage device 1200. The information processing apparatus 1300 can cause patient information and information relating to photoacoustic image data pieces to be displayed on the lists 1440 and 1450 with reference to their incidental information. A list 1460 is a list of candidates for composition image data requested to be computed.

As illustrated in FIG. 4, according to this embodiment, a case will be described in which a user uses the input device 1500 to designate oxygen saturation as composition image data from the list 1460. It should be noted that the display mode of the item corresponding to the designated oxygen saturation may be changed (such as a thick frame of the item in FIG. 4).

### S400: Processing for Displaying Combination of Photoacoustic Image Data Pieces Adaptable for Computing Composition Image Data

The information processing apparatus 1300 as an incidental information obtaining unit is configured to obtain incidental information of photoacoustic image data set stored in the storage device 1200. The information processing apparatus 1300 as a determining unit is configured to determine a combination of photoacoustic image data pieces adaptable for computing composition image data of a type indicated by the type information with reference to the incidental information of the photoacoustic image data set stored in the storage device 1200.

For example, the information processing apparatus 1300 may determine a combination of photoacoustic image data pieces with respective photographed dates and times included in a predetermined period as a combination adaptable for computing composition image data. The information processing apparatus 1300 may determine a combination of photoacoustic image data pieces with a difference between the respective photographed dates and times satisfying a predetermined condition as a combination adaptable for computing composition image data. In order to compute a concentration of a substance in composition image data, the information processing apparatus 1300 may select a combination of photoacoustic image data pieces with a difference between respective photographed dates and times equal to or lower than a predetermined threshold value or with the smallest difference between the photographed dates and times. Alternatively, in order to compute difference information between composition image data pieces, the information processing apparatus 1300 may select a combination of photoacoustic image data pieces with a difference between the respective photographed dates and times in a predetermined follow-up period.

Alternatively, the information processing apparatus 1300 may determine a combination of photoacoustic image data pieces based on an identical photographed region as combination adaptable for computing composition image data.

The information processing apparatus 1300 determines a combination of photoacoustic image data pieces with respective measured wavelengths adaptable for computing composition image data as a combination adaptable for computing composition image data. For example, for computing an oxygen saturation as composition image data, a combination of photoacoustic image data pieces corresponding to measured wavelengths different from each other from a range of 700 nm through 1000 nm. For computing an oxygen saturation as composition image data, the information processing apparatus 1300 may select photoacoustic image data pieces of an image type being an absorption coefficient distribution. For computing an oxygen saturation as composition image data, the information processing apparatus 1300 may select photoacoustic image data pieces of an identical image type.

For example, a case will be examined in which the type indicated by the type information is concentration information of a substance contained in an object, which is computed from a plurality of photoacoustic image data pieces by applying light having a plurality of wavelengths to the object. The information processing apparatus 1300 determines whether given photoacoustic image data pieces are of an identical patient with reference to respective patient information pieces given as incidental information. For example, the information processing apparatus 1300 determines photoacoustic image data pieces including an identical patient ID as incidental information from photoacoustic image data set with reference to the patient ID associated as the incidental information of the photoacoustic image data set. The information processing apparatus 1300 determines photoacoustic image data pieces of an identical photographed region from photoacoustic image set with reference to information regarding the photographed regions associated as incidental information of the photoacoustic image data set. Alternatively, the information processing apparatus 1300 determines photoacoustic image data pieces photographed with light having wavelengths different from each other from photoacoustic image data set with reference to information on the wavelengths of irradiation light associated as incidental information of the photoacoustic image data set. The information processing apparatus 1300 determines photoacoustic image data pieces of an identical image type from photoacoustic image data set with reference to information on image types associated as incidental information of the photoacoustic image data set. The information processing apparatus 1300 may determine photoacoustic image data pieces having a image type that is an optical absorption coefficient distribution from photoacoustic image data set with reference to information on the image types associated as incidental information of the photoacoustic image data set. The information processing apparatus 1300 may determine photoacoustic image data pieces of an image type that is an optical absorption coefficient distribution from photoacoustic image data set with reference to information regarding image types associated as incidental information of the photoacoustic image data set. Image types excluding an optical absorption coefficient distribution may be used to compute concentration information of a substance contained in an object, which disadvantageously results in a lower quantative property. Against the disadvantage, photoacoustic image data pieces of an image type that is an optical absorption coefficient distribution may be selectively used to accurately compute concentration information on a substance contained in an object. Alternatively, the information processing apparatus 1300 determines photoacoustic image data pieces photographed at a same date from the photoacoustic image data set with reference to information regarding photographed dates and times associated as incidental information of the photoacoustic image data set. This is because there is a possibility that the concentration of a substance contained in the object may be different between photoacoustic image data pieces photographed at different dates. Therefore, it is difficult to compute with high accuracy the concentration information on a substance contained in the object when photoacoustic image data pieces photographed at different dates are used. On the other hand, use of a plurality of photoacoustic images photographed at a same date enables to compute with high accuracy concentration information of a substance included in the object. Then, the information processing apparatus 1300 determines a combination of photoacoustic image data pieces determined as satisfying one of the conditions above as a combination adaptable for computing composition image data. It should be noted that criteria when concentration information is designated as resulting composition image data are not limited thereto. In this case, the information processing apparatus 1300 may determine photoacoustic image data pieces at least photographed with wavelengths different from each other and of an identical image type. The information processing apparatus 1300 may sequentially narrow photoacoustic image data pieces satisfying the conditions in the photoacoustic image data set. For example, photoacoustic image data pieces regarding an identical patient are determined in a photoacoustic image data set, and photoacoustic image data pieces photographed with light having wavelengths different from each other in the photoacoustic image data set regarding the identical patient to determine a combination of photoacoustic image data pieces.

The information processing apparatus 1300 can determine photoacoustic image data pieces adaptable for computing composition image data of a type indicated by type information with reference to a table illustrating a relationship between composition image data pieces of a plurality of types and conditions for photoacoustic image data pieces adaptable for computing the types. When photoacoustic image data pieces adaptable for computing composition image data of type indicated by the type information can be determined, any other methods excluding the method with reference to the table can be applied.

The information processing apparatus 1300 as a display control unit outputs information indicating a combination of photoacoustic image data pieces adaptable for computing composition image data to the display apparatus 1400. The display apparatus 1400 can display the information indicating a combination of photoacoustic image data pieces adaptable for computing composition image data. Any method is applicable for displaying information indicating a combination of photoacoustic image data pieces adaptable for computing composition image data. For example, the information processing apparatus 1300 causes to display a list of combinations of photoacoustic image data pieces adaptable for computing composition image data. The list may undergo sorting with reference to patient information as incidental information or information regarding photoacoustic image data pieces. On a list of photoacoustic image data pieces, adaptable data for computing composition image data and inadaptable data may be displayed in different display modes from each other. The different display modes can be implemented by different text color of text describing data or by hiding inadaptable data.

It should be noted that one combination or a plurality of combinations of photoacoustic image data pieces may be output to the display apparatus 1400. The information processing apparatus 1300 may obtain information indicating a combination of photoacoustic image data pieces that is inadaptable for computing composition image data and output it to the display apparatus 1400.

The information processing apparatus 1300 as an adaptability acquiring unit may acquire an adaptability which indicates whether a combination of photoacoustic image data pieces is adaptable. The information processing apparatus 1300 may set an adaptability of 1 in a case where a combination of photoacoustic image data pieces is the most adaptable for computing composition image data and set an adaptability of 0 in a case where a combination of photoacoustic image data pieces is least adaptable for computing composition image data. The adaptabilities may be represented in a stepwise manner. For example, the information processing apparatus 1300 may determine adaptabilities in a stepwise manner in accordance with the number of satisfactions of conditions for photoacoustic image data pieces adaptable for computing composition image data. As the number of satisfactions of the conditions increases, the level of the adaptability may increase. A weight to be given to the adaptabilities may be changed in accordance with the conditions. In a case where composition image data is concentration information of a substance contained in an object, weights for the condition that photoacoustic image data pieces are photographed with light having wavelengths different from each other, and the weight for the condition may be higher than those for other conditions. The determining of a combination of photoacoustic image data pieces adaptable for computing composition image data corresponds to acquisition of an adaptability indicating whether the combination of photoacoustic image data pieces is adaptable for computing composition image data. The information processing apparatus 1300 can cause the display apparatus 1400 to display information based on the adaptability.

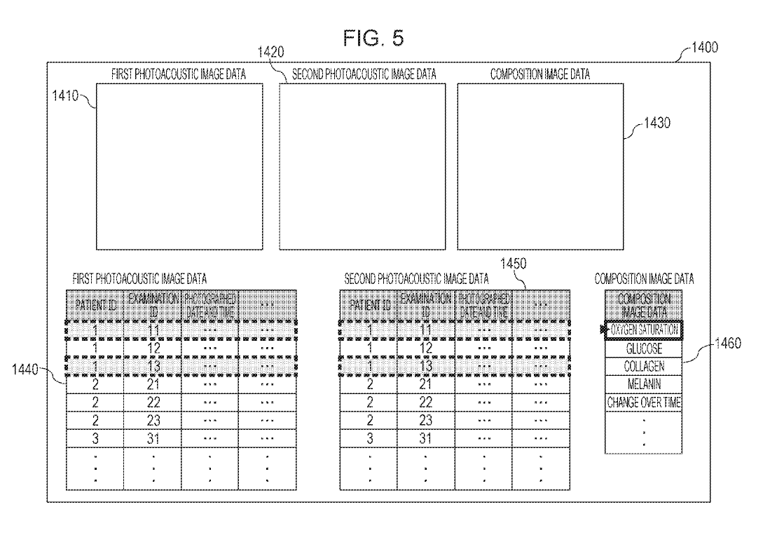

As illustrated in FIG. 5, the information processing apparatus 1300 causes to display a plurality of item so as to differentiate a display mode for items corresponding to a combination of photoacoustic image data pieces adaptable for computing an oxygen saturation and display modes for other items. That is, the information processing apparatus 1300 differentiates the display mode of the items corresponding to a adaptable combination of a photoacoustic image data pieces that is a list 1440 in the photoacoustic image data set displayed in the list 1450 and the display modes for other items. Referring to the example illustrated in FIG. 5, items corresponding to adaptable combinations of photoacoustic image data pieces are placed within a broken line frame and have a background color that is different from other items. Thus, a user can identify items corresponding to a combination of photoacoustic image data pieces adaptable for computing an oxygen saturation.

### S500: Processing for Designating First Photoacoustic Image Data Piece

A user may use the input device 1500 to designate first photoacoustic image data (first photoacoustic image data piece) for computing composition image data from photoacoustic image data set stored in the storage device 1200. For example, a user can designate a desired photoacoustic image data piece from a list of photoacoustic image data pieces displayed on the display apparatus 1400. Any method is applicable for designating a first photoacoustic image data piece from the photoacoustic image data set. The information processing apparatus 1300 as an designation information obtaining unit obtains designation information defining the first photoacoustic image data piece designated based on an instruction by a user through the input device 1500. The information processing apparatus 1300 obtains incidental information of the first photoacoustic image data defined by the designation information. In other words, the information processing apparatus 1300 obtains the incidental information of the first photoacoustic image data piece by reading out them from the storage device 1200 based on the designation information.

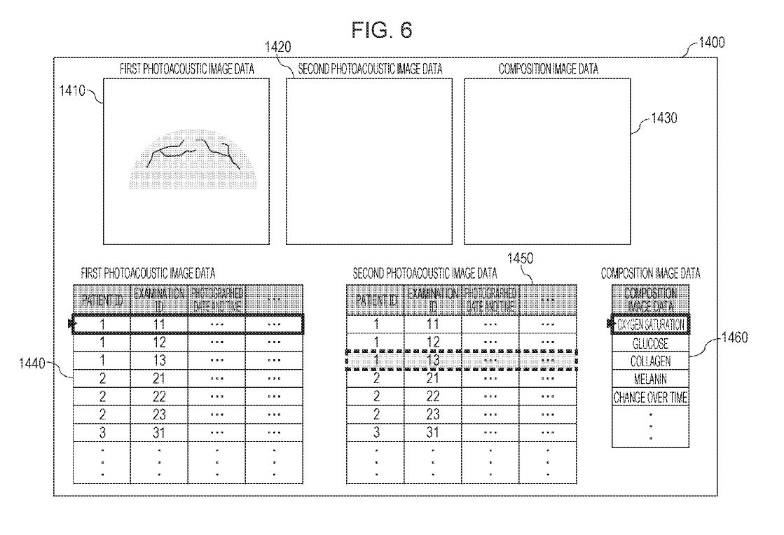

FIG. 6 illustrates a case where an item corresponding to a photoacoustic image data piece with a patient ID 1 and an examination ID 11 on the list 1440 as the first photoacoustic image data piece. The information processing apparatus 1300 causes the image based on the first photoacoustic image data piece designated by the user to be displayed on the display region 1410. Thus, the user can check whether the designated photoacoustic image data piece is a desired image data piece.

### S600 Processing for Determining Whether Designated First Photoacoustic Image Data Piece is Adaptable

The information processing apparatus 1300 determines whether the first photoacoustic image data piece is adaptable for computing composition image data of a type indicated by the type information based on the type information and the incidental information of the first photoacoustic image data piece. In other words, the information processing apparatus 1300 acquires an adaptability of the first photoacoustic image data piece for composition image data. It should be noted that the information processing apparatus 1300 may start computing an adaptability when a photoacoustic image data piece is selected or when an icon for starting computation of the adaptability displayed on the display apparatus 1400 is selected.

In a case where the information processing apparatus 1300 judges that the adaptability of the designated first photoacoustic image data piece is low, that is, the designated first photoacoustic image data piece is not adaptable for computing composition image data, the information processing apparatus 1300 causes the display apparatus 1400 to display the fact (S700). Further, a reason why the designated first photoacoustic image data piece is inadaptable may be displayed such as the designated photoacoustic image data piece does not have a desired measured wavelength.

In a case where adaptabilities are represented in a stepwise manner, the information processing apparatus 1300 judges that a first photoacoustic image data piece having an adaptability higher than a threshold value is adaptable for computing composition image data. The information processing apparatus 1300 may judge that a first photoacoustic image data piece having an adaptability lower than the threshold value is not adaptable for computing composition image data.

### S800: Processing for Displaying Second Photoacoustic Image Data Piece Adaptable for Computing Composition Image Data

The information processing apparatus 1300 determines another photoacoustic image data adaptable for computing composition image data in a case where the designated first photoacoustic image data is judged as being adaptable for computing composition image data. The information processing apparatus 1300 determines another photoacoustic image data piece stored in the storage device 1200, which is adaptable for computing composition image data based on the type information, designation information defining the first photoacoustic image data piece, and incidental information of the photoacoustic image data set stored in the storage device 1200. For computing composition image data, the information processing apparatus 1300 determines second photoacoustic image data (second photoacoustic image data piece) that is adaptable for a combination with the first photoacoustic image data piece. The information processing apparatus 1300 outputs information regarding the second photoacoustic image data piece adaptable for computing composition image data to the display apparatus 1400 and causes the display apparatus 1400 to display the information regarding the second photoacoustic image data piece adaptable for computing composition image data. The information processing apparatus 1300 may output to the display apparatus 1400 the adaptability for a combination as well as the adaptability of the second photoacoustic image data for computing composition image data.

The second photoacoustic image data piece adaptable for computing composition image data may be determined under the same conditions as those for determination of the combination of photoacoustic image data pieces adaptable for computing composition image data.

As illustrated in FIG. 6, in a case where an oxygen saturation is designated as composition image data and an item with the examination ID 11 is designated as a first photoacoustic image data piece, the item with a examination ID 13 in a list 1450 is displayed in a different display mode. Thus, a user can easily grasp candidates for the second photoacoustic image data that is adaptable for computing an oxygen saturation and that is adaptable for a combination with the first photoacoustic image data.

### S900: Processing for Designating Second Photoacoustic Image Data Piece

A user may use the input device 1500 to designate a second photoacoustic image data (second photoacoustic image data piece) to be used for computing composition image data from the photoacoustic image data set stored in the storage device 1200. For example, a user may designate a desired photoacoustic image data piece from a list of photoacoustic image data pieces displayed on a display apparatus 1400. The second photoacoustic image data may be designated from photoacoustic image data set by any method. The information processing apparatus 1300 obtains designation information defining the second photoacoustic image data piece designated based on a user's instruction through the input device 1500. The information processing apparatus 1300 obtains incidental information of the second photoacoustic image data piece defined by the designation information. In other words, the information processing apparatus 1300 obtains the incidental information of the second photoacoustic image data piece by reading out it through the storage device 1200 based on the designation information.

### S1000: Processing for Determining Whether Designated Combination of Photoacoustic Image Data Pieces is Adaptable

Based on the type information and the incidental information of the first and second photoacoustic image data pieces, the information processing apparatus 1300 determines whether the combination of photoacoustic image data pieces is adaptable for computing composition image data of a type indicated by type information. In other words, the information processing apparatus 1300 acquires the adaptability of the combination of the first and second photoacoustic image data pieces for composition image data. The information processing apparatus 1300 may start the computing of an adaptability when two photoacoustic image data pieces are selected or after an icon for starting the computing of an adaptability, which is displayed on the display apparatus 1400, is selected.

In a case where the designated combination of first and second photoacoustic image data pieces has a low adaptability, the information processing apparatus 1300 causes the display apparatus 1400 to display the fact (S1100). In other words, in a case where the information processing apparatus 1300 judges that the designated combination of first and second photoacoustic image data pieces is not adaptable for computing composition image data, the display apparatus 1400 is caused to display the fact. In a case where the designated combination of first and second photoacoustic image data pieces has a low adaptability, a reason why the designated combination of first and second photoacoustic image data pieces is inadaptable may be displayed such as because the designated photoacoustic image data has a measured wavelength that is not desirable. In a case where the designated combination of first and second photoacoustic image data pieces has a low adaptability, the processing is controlled such that the information processing apparatus 1300 is prevented from executing the computing of composition image data. In other words, in a case where the designated combination of first and second photoacoustic image data pieces has a low adaptability, the information processing apparatus 1300 may be controlled so as not to receive an instruction to compute composition image data from a user.

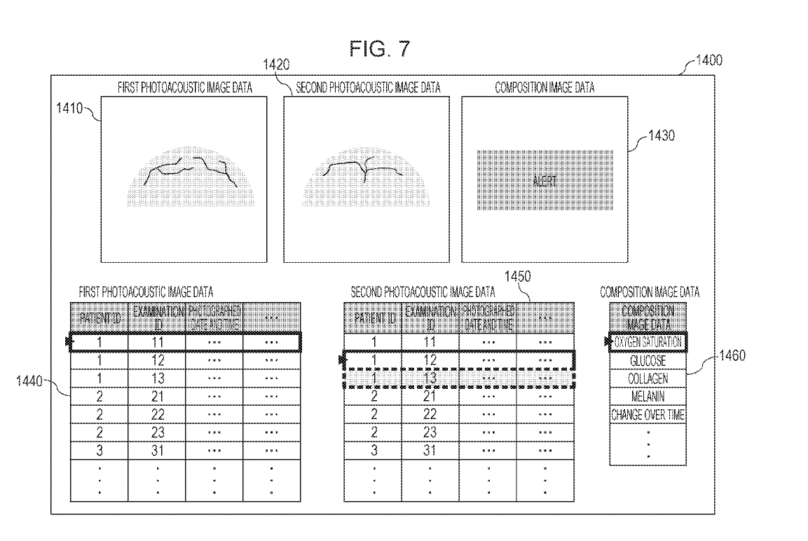

FIG. 7 illustrates a case where a photoacoustic image data piece corresponding to an examination ID 12 is designated, which is not adaptable for a combination with the photoacoustic image data corresponding to the examination ID 11 for computing an oxygen saturation in S400. In this case, the information processing apparatus 1300 causes a display region 1430 to display an alert indicating that the photoacoustic image data corresponding to the examination ID 12 is not adaptable for computing an oxygen saturation. The region for displaying such an alert is not limited to the display region 1430 but may be any region. According to this embodiment, the information processing apparatus 1300 causes a display region 1420 to display a photoacoustic image data corresponding to an item selected from the list 1450. The information processing apparatus 1300 may display the item corresponding to the examination ID 13, which is determined as being adaptable for the combination in the list 1450, in a display mode different from those for the other items.

### S1200: Processing for Computing Composition Image Data

The information processing apparatus 1300 as a composition image data computing unit computes and generates composition image data by using first and second photoacoustic image data pieces in a case where the designated combination of the first and second photoacoustic image data pieces has a high adaptability. In other words, in a case where the designated first and second photoacoustic image data pieces are judged as being adaptable for computing composition image data, the information processing apparatus 1300 computes and generates the composition image data. The information processing apparatus 1300 uses the first and second photoacoustic image data pieces designated by a user to compute the composition image data. It should be noted that the information processing apparatus 1300 may transmit information indicating the adaptability to the display apparatus 1400 and may cause the display apparatus 1400 to display that the combination of first and second photoacoustic image data pieces is adaptable for computing composition image data. The information processing apparatus 1300 may start the processing for computing composition image data in response to an instruction from a user after an indication based on an adaptability is displayed. Thus, a user can check the adaptability of a combination of image data pieces and, if the user judges that there is no problem, can instruct to start the computing processing. In a case where there is one combination of photoacoustic image data pieces adaptable for computing composition image data in S400, the information processing apparatus 1300 may be perform the processing in S500 to S1100. In this case, the information processing apparatus 1300 may compute composition image data by using photoacoustic image data pieces corresponding to the combination determined in S400. The information processing apparatus 1300 may output the computed composition image data to the storage device 1200 for storage. The information processing apparatus 1300 may output the computed composition image data to the display apparatus 1400 and causes the image based on the composition image data to be displayed.

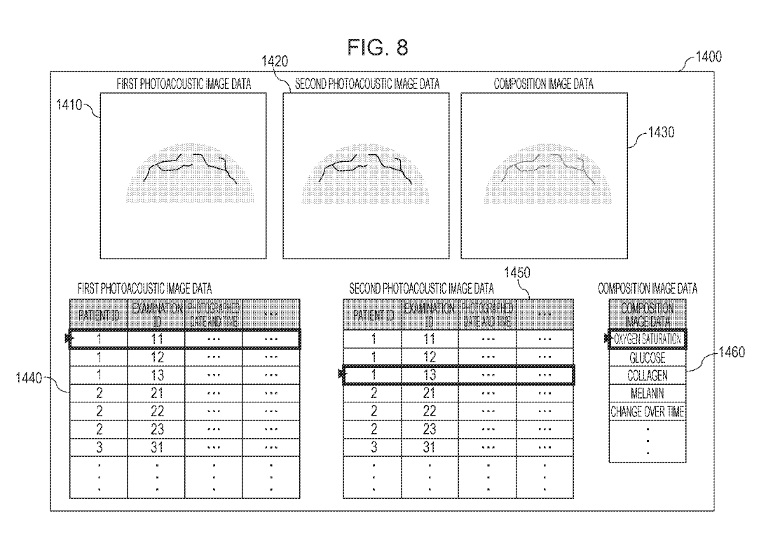

FIG. 8 illustrates a case where a photoacoustic image data piece corresponding to the examination ID 11 and a photoacoustic image data corresponding to an examination ID 13 are designated as a first photoacoustic image data and a second photoacoustic image data, respectively. Assume that the combination is determined as being adaptable in S400. In this case, the information processing apparatus 1300 uses the photoacoustic image data piece corresponding to the examination ID 11 and the photoacoustic image data piece corresponding to the examination ID 13 to compute an oxygen saturation and causes the display region 1430 to display an image indicating a space distribution of the oxygen saturation. The composition image data displayed here is highly possibly accurate information because the information is computed based on a combination of photoacoustic image data pieces determined as being adaptable with reference to the incidental information of the photoacoustic image data pieces.

Having described the example, according to this embodiment, in which a user designates two photoacoustic image data pieces sequentially, the method for designating photoacoustic image data pieces to be used for computing composition image data is not limited to the method. A combination displayed in S400 may be designated to designate a plurality of photoacoustic image data pieces corresponding to the combination. Alternatively, the number of photoacoustic image data pieces to be used for computing composition image data is not limited to two, but three or more photoacoustic image data pieces may be designated so that the three or more photoacoustic image data pieces can be used to compute composition image data.

Having described the example, according to this embodiment, in which the system includes the photoacoustic apparatus 1100 configured to generate photoacoustic image data, embodiments of the present disclosure are also applicable to a system not including the photoacoustic apparatus 1100. Embodiments of the present disclosure are applicable to any system including the information processing apparatus 1300 which can obtain photoacoustic image data. Embodiments of the present disclosure is applicable to a system including the storage device 1200 and the information processing apparatus 1300 and excluding the photoacoustic apparatus 1100, for example. In this case, the information processing apparatus 1300 can obtain photoacoustic image data by reading out designated photoacoustic image data pieces from photoacoustic image data set prestored in the storage device 1200.

Any method can complement the designation of photoacoustic image data pieces when photoacoustic image data pieces that are adaptable for computing composition image data can be designated based on type information defining the type of the composition image data requested to be computed and incidental information of the photoacoustic image data pieces in the storage device 1200. For example, processing can be performed in order of S400→S500→S900→S1200 so that a user can designate a plurality of photoacoustic image data pieces with reference to the combination adaptable for computing composition image data. Performing the processing may perform in order of S500→S600→S700→S500 and so on can reduce the possibility of a user that a first photoacoustic image data that is inadaptable for computing composition image data is unintentionally designated. The processing may be performed in order of S8800→S900 so that a user can be facilitated to designate a second photoacoustic image data piece that is adaptable for computing composition image data and adaptable for a combination with the first photoacoustic image data piece. Performing the processing may be performed in order of S500→S900→81000→S1100→S1000 can reduce the possibility that a combination of photoacoustic image data pieces that are not adaptable for computing composition image data is designated. In all of the examples above, with the information processing apparatus according to this embodiment, a user can be facilitated to designate photoacoustic image data that is adaptable for computing composition image data.

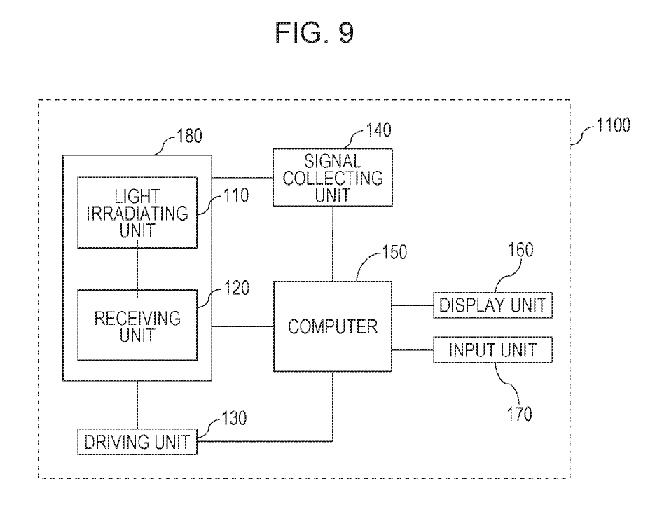

Next, an example of a configuration of apparatuses included in the system according to this embodiment will be described. FIG. 9 is a schematic block diagram illustrating apparatuses included in the system according to this embodiment.

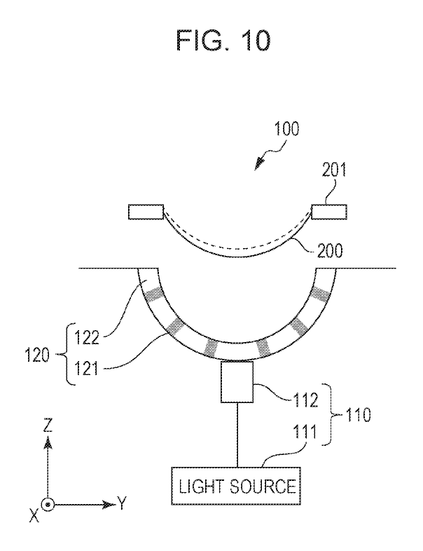

The photoacoustic apparatus 1100 according to this embodiment has a driving unit 130, a signal collecting unit 140, a computer 150, and a probe 180. The probe 180 has a light irradiating unit 110, and a receiving unit 120. FIG. 10 is a schematic diagram of the probe 180 according to this embodiment. An object 100 is to be measured. The driving unit 130 is configured to drive the light irradiating unit 110 and the receiving unit 120 to perform mechanical scanning. The object 100 is irradiated with light by the light irradiating unit 110, and acoustic waves are generated within the object 100. The acoustic waves generated by a photoacoustic effect due to the light will also be called photoacoustic waves. The receiving unit 120 is configured to receive the photoacoustic waves and to output an electric signal (photoacoustic signal) as an analog signal.

The signal collecting unit 140 is configured to convert the analog signal output from the receiving unit 120 to a digital signal and to output it to the computer 150. The computer 150 is configured to store the digital signal output from the signal collecting unit 140 as signal data originating from the photoacoustic waves.

The computer 150 is configured to perform signal processing on the digital signal stored therein to generate photoacoustic image data. The computer 150 is further configured to perform image processing on the acquired photoacoustic image data and to output the photoacoustic image data to a display unit 160. The display unit 160 is configured to display a photoacoustic image based on the photoacoustic image data. The display image is stored in a memory within the computer 150 or the storage device 1200 of a data management system connected to modality over a network in response to a storage instruction from a user or the computer 150.

The computer 150 is further configured to control driving of components included in the photoacoustic apparatus. The display unit 160 may display an image generated by the computer 150 and a GUI, for example. The input unit 170 is configured to be usable by a user for inputting information. A user can perform operations such as a start and a stop of a measurement, an instruction to store a generated image and so on.

Details of components of the photoacoustic apparatus 1100 according to this embodiment will be described below.

### Light Irradiating Unit 110

The light irradiating unit 110 includes a light source 111 configured to emit light and an optical system 112 configured to guide the light emitted from the light source 111 to the object 100. The light here includes pulsed light of so-called square waves or triangle waves.

The light emitted from the light source 111 has a pulse width equal to or higher than 1 ns and equal to or lower than 100 ns. The light may have a wavelength of a range about 400 nm to 1600 nm. For imaging a blood vessel at a high resolution, a wavelength (equal to or higher than 400 nm and equal to or lower than 700 nm) may be used which can be significantly absorbed by the blood vessel. For imaging a deep part of a living body, a wavelength (equal to or higher than 700 nm and equal to or lower than 1100 nm) may be used which can be typically less absorbed by background tissue (of water or fat) of a living body.

The light source 111 may be a laser or a light emitting diode. The light source may emit light with a changeable wavelength for measuring with light having a plurality of wavelengths. In a case where an object is to be irradiated with light having a plurality of wavelengths, a plurality of light sources may be prepared which generate light beams having wavelengths different from each other, and light beams are emitted from the light sources alternately. A plurality of light sources if used is called a light source collectively. The laser may vary such as a solid-state laser, a gas laser, a dye laser, a semiconductor laser. For example, the light source may be a pulsed laser such as an Nd:YAG laser or a alexandrite laser. Alternatively, the light source may be T:sa laser or an OPO (optical parametric oscillators) laser with an Nd:YAG laser as excited light. The light source 111 may be a flash lamp or a light emitting diode. The light source 111 may be a microwave source.

The optical system 112 may include optical elements such as a lens, a mirror, and a optical fiber. In a case where a breast, for example, is the object 100, the light emitting unit in the optical system may have a diffusing plate configured to diffuse light so that the object 100 can be irradiated with pulsed light having an increased beam diameter. For an increased resolution, on the other hand, realized with a photoacoustic microscope, the light emitting unit of the optical system 112 may include a lens so that focused beam can be irradiated.

It should be noted that the object 100 is directly irradiated with light from the light source 111 without the optical system 112 in the light irradiating unit 110.

### Receiving Unit 120

The receiving unit 120 includes a transducer 121 configured to output an electric signal by receiving acoustic waves and a supporting member 122 configured to support the transducer 121. The transducer 121 may be configured to transmit acoustic waves as a transmitting unit. The transducer as a receiving unit and the transducer as a transmitting unit may be implemented by a single (common) transducer or may be separate components.

The transducer 121 may contain members of a piezoelectric ceramic material such as a PZT (lead zirconate titanate) and a high molecule piezoelectric film material such as a PVDF (poly(vinylidene fluoride)). An element excluding such a piezoelectric element may be used. For example, a transducer may be used such as capacitive micro-machined ultrasonic transducers (CMUT). It should be noted that any transducer is applicable when it can receive acoustic waves and thus output an electric signal. Furthermore, a time-resolved signal is acquired by the transducer. In other words, the amplitude of the signal acquired by the transducer represents a value based on sound pressure (or a value in proportion to a sound pressure, for example) received by the transducer at each clock time.

The photoacoustic waves may contain a frequency component in a range from, typically, 100 KHz to 100 MHz, and the transducer 121 may further be configured to detect such frequencies.

The supporting member 122 may be formed from a metallic material having a high mechanical strength. A side surface of the supporting member 122 to be closer to the object 100 may be mirror-like finished or be processed to scatter light so that more irradiation light can be incident on the object. According to this embodiment, the supporting member 122 has a hemispherical shell shape such that a plurality of transducers 121 can be supported on the hemispherical shell. In this case, the directional axes of the transducers 121 arranged on the supporting member 122 gather around the curvature center of the hemisphere. Signals output from a plurality of transducer 121 are used for higher image quality around the curvature center when imaged. Any supporting member 122 may be used if it can support the transducers 121. The supporting member 122 may have a plurality of transducers on its plane or curved surface to obtain a 1D array, a 1.5D array, a 1.75D array, or 2D array, for example. The plurality of transducers 121 corresponds to a plurality of receiving units.

The supporting member 122 may function as a container for retaining an acoustic matching material. In other words, the supporting member 122 may be a container for arranging an acoustic matching material between the transducers 121 and the object 100.

The receiving unit 120 may have an amplifier configured to amplify time-series analog signals output from the transducer 121. The receiving unit 120 may have an A/D converter configured to convert time-series analog signals output from the transducer 121 to time-series digital signals. In other words, the receiving unit 120 may have the signal collecting unit 140, which will be described below.

A space between the receiving unit 120 and the object 100 is filled with a medium which can propagate photoacoustic waves. The medium may have a material to which acoustic waves can be propagated, having an interface with the object 100 or the transducer 121 where the acoustic properties are matched and exhibiting a transmittance of photoacoustic waves as high as possible. For example, the medium may be water or an ultrasonic wave gel.

FIG. 10 is an elevation diagram of the probe 180. The probe 180 according to this embodiment has a receiving unit 120 having a plurality of transducers 121 arranged three-dimensionally on the supporting member 122 having a shape of hemisphere with an aperture. The supporting member 122 further has a light emitting unit of the optical system 112 at its bottom.

According to this embodiment, the object 100 is in contact with the holding unit 200 as illustrated in FIG. 10 so that its shape can be held.

A space between the receiving unit 120 and the holding unit 200 is filled with a medium which can propagate photoacoustic waves. The medium may have a material to which photoacoustic waves can be propagated, having an interface with the object 100 or the transducer 121 where the acoustic properties are matched and exhibiting a transmittance of photoacoustic waves as high as possible. For example, the medium may be water or an ultrasonic wave gel.

The holding unit 200 as a holding unit is used for holding shapes of the object 100 while being measured. The holding unit 200 holds the object 100 to prevent movements of the object 100 and hold the position of the object 100 within the holding unit 200. The holding unit 200 may contain a resin material such as polycarbonate, polyethylene, polyethylene terephthalate.

The holding unit 200 has an attachment unit 201 attached. The attachment unit 201 may enable to replace the holding unit 200 by one of a plurality of types of holding unit 200 in accordance with the size of a given object. For example, the attachment unit 201 may enable replacement by a holding unit having a curvature radius or a curvature center different from that of the currently attached holding unit.

### Driving Unit 130

The driving unit 130 is configured to change the relative position between the object 100 and the receiving unit 120. The driving unit 130 includes a motor such as a stepping motor configured to generate driving force, a driving mechanism configured to transmit the driving force, and a position sensor configured to detect positional information regarding the receiving unit 120. The driving mechanism may be a leading screw mechanism, a link mechanism, a gear mechanism, a hydraulic mechanism, or the like. The position sensor may be a potentiometer based on an encoder, a variable resistor, a linear scale, a magnetic sensor, an infrared sensor, an ultrasonic sensor or the like.

The driving unit 130 is not limited to those which change the relative positions of the object 100 and the receiving unit 120 in X and Y directions (two-dimension) but may be one which changes the relative positions one- or three-dimensionally.

The driving unit 130 may have the receiving unit 120 at a fixed position and move the object 100 if the relative positions of the object 100 and the receiving unit 120 can be changed. In order to move the object 100, the holding unit holding the object 100 may be moved to move the object 100. Both of the object 100 and the receiving unit 120 can be moved.

The driving unit 130 may move the relative position continuously or in a step-and-repeat manner. The driving unit 130 may be an electric stage configured to move on a programmed path or a manual stage.

According to this embodiment, the driving unit 130 drives the light irradiating unit 110 and the receiving unit 120 simultaneously to scan. However, the driving unit 130 may move the light irradiating unit 110 or the receiving unit 120 only.

### Signal Collecting Unit 140

The signal collecting unit 140 includes an amplifier and an A/D converter. The amplifier is configured to amplify electric signals that are analog signals output from the transducers 121. The A/D converter is configured to convert the analog signals output from the amplifier to digital signals. The digital signals output from the signal collecting unit 140 are stored in a storage unit 152 within the computer 150. The signal collecting unit 140 is also called a data acquisition system (DAS). The electric signal herein is a concept including an analog signal and a digital signal. It should be noted that an optical detection sensor may detect light emission from the light irradiating unit 110, and the signal collecting unit 140 may start the processing above in synchronism with a trigger of the detection result.

### Computer 150

The computer 150 is configured by the same hardware as that of the information processing apparatus 1300. In other words, a unit responsible for the computing function of the computer 150 can include a processor such as a CPU or a GPU (Graphics Processing Unit) and a computing circuit such as an FPGA (Field Programmable Gate Array) chip. These units may include a single processor and a single computing circuit but instead may include a plurality of processors and computing circuits.

A unit responsible for the storage function of the computer 150 may be a volatile medium such as a RAM (random access memory). It should be noted that a storage medium configured to store a program is a non-transitory storage medium. The unit responsible for the storage function of the computer 150 may include one storage medium but instead may include a plurality of storage media.

A unit responsible for the control function of the computer 150 may include a computing element such as a CPU. A unit responsible for the control function of the computer 150 is configured to control actions of components of the photoacoustic apparatus. A unit responsible for the control function of the computer 150 may control the components of the photoacoustic apparatus in response to an instruction signal by an operation for, for example, starting a measurement through the input unit 170. A unit responsible for the control function of the computer 150 is configured to read out program code stored in a unit responsible for the storage function and control operations of components of the photoacoustic apparatus.

### Display Unit 160

The display unit 160 is a display apparatus such as a liquid crystal display or an organic electro luminescence. The display unit 160 may further display a GUI for operating an image or an apparatus.

### Input Unit 170

The input unit 170 may be an operating console including a mouse and a keyboard, for example, which can be operated by a user. The display unit 160 may be a touch panel so that the display unit 160 can also be used as the input unit 170.

### Object 100

Although the object 100 is not a component of the photoacoustic apparatus, it will be described below. A photoacoustic apparatus according to the following embodiment is mainly usable for a diagnosis and a follow-up study of a chemical treatment performed on a malignant tumor or a blood vessel disease of a human or an animal. Therefore, the object 100 may be a living body, and, more specifically, it may be a diagnosis target region such as the breast, the internal organs, vascular networks, the head, the neck, the abdomen or the limbs including fingers and toes of a human or animal body. For example, in a case where a human body is a measurement target, the target of the light absorber may be oxyhemoglobin or deoxyhemoglobin or a blood vessel containing a large amount of them or a malignant tumor containing many neovessels. Plaque of a carotid artery wall may be an optical absorber. Melanin, collagen, collagen, glucose, or lipid contained in the skin may be an optical absorber. Alternatively, a pigment such as methylene blue (MB), indocyanine green (ICG), gold minute particles, or an externally introduced substance integrating or chemically modifying them may be an optical absorber. A phantom imitating a living body may be the object 100.

The components of the photoacoustic apparatus may be provided as separate devices or may be integrated into one apparatus. At least some components of the photoacoustic apparatus may be integrated as one device.

The apparatuses included in the system according to this embodiment may be implemented by separate hardware modules, or all of the apparatuses may be implemented by one hardware module. The functions of the system according to this embodiment may be any hardware modules.

### Flow for Generating Photoacoustic Image Data

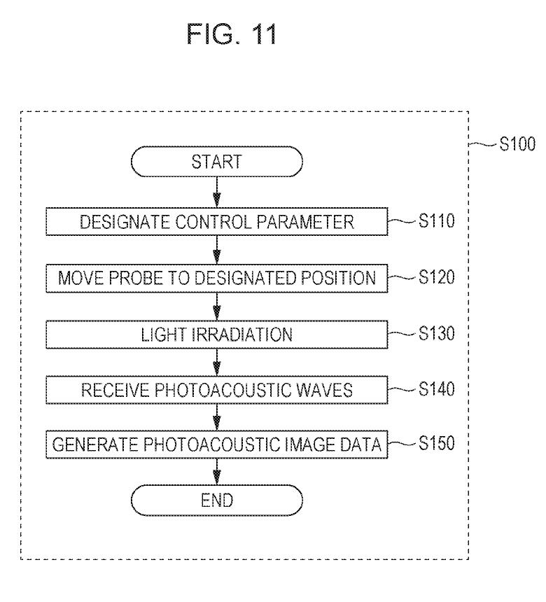

Next, processes for generating photoacoustic image data to be performed by the photoacoustic apparatus 1100 will be described with reference to FIG. 11.

### S110: Process for Designating Control Parameter

A user may designate a control parameter such as irradiation conditions (such as a repetition frequency and a wavelength) for the light irradiating unit 110 for acquiring object information and positions of the probe 180 by using the input unit 170. The computer 150 sets the determined control parameters based on a user's instruction.

### S120: Process for Moving Probe to Designated Position

A control unit 153 moves causes the driving unit 130 to move the probe 180 to a designated position based on the control parameter designated in S110. When photographing at a plurality of positions is designated in S110, the driving unit 130 first moves the probe 180 to a first designated position. It should be noted that the driving unit 130 may move the probe 180 to a position that is programmed in advance when a measurement start instruction is given.

S130: Process for Irradiating with Light

The object 100 is irradiated with light by the light irradiating unit 110 based on the control parameters designated in S110.

The object 100 is irradiated with the light emitted from the light source 111 as pulsed light through the optical system 112. The pulsed light is absorbed within the object 100 and photoacoustic waves occur because of the photoacoustic effect. The light irradiating unit 110 transmits pulsed light and also transmits a synchronizing signal to the signal collecting unit 140.

### S140: Process for Receiving Photoacoustic Waves

When the signal collecting unit 140 receives the synchronizing signal transmitted from the light irradiating unit 110, the signal collecting unit 140 starts an operation for signal collection. In other words, the signal collecting unit 140 amplifies and AD converts analog electric signals originating from the acoustic waves output from the receiving unit 120 to generate the amplified digital electric signals and output them to the computer 150. The computer 150 stores the signals transmitted from the signal collecting unit 140 in the storage unit 152. When photographing at a plurality of scan positions is designated in S110, the processing in steps S120 to S140 are repeatedly performed at the designated scan positions and repeatedly irradiate pulsed light generate digital signals originating from the acoustic waves. It should be noted that light emission may trigger the computer 150 to obtain and store the positional information on the receiving unit 120 upon the light emission based on an output from the position sensor in the driving unit 130.

### S150: Process for Generating Photoacoustic Image Data

A computing unit 151 in the computer 150 as an image generating unit generates photoacoustic image data based on signal data stored in the storage unit 152. The computer 150 outputs the generated photoacoustic image data to the storage device 1200 which then stores the data.

A reconstruction algorithm for converting signal data to volume data as a space distribution may be an analytical reconfiguration method such as back projection in a time domain or back projection in a Fourier domain or model-based method (repeated calculation method). For example, the back projection in a time domain may be a Universal back-projection (UBP), a Filtered back-projection (FBP), or phasing addition (Delay-and-Sum).

The computing unit 151 may compute a light fluence distribution within the object 100 of the light applied to the object 100 and divides an initial sound pressure distribution by the light fluence distribution to acquire absorption coefficient distribution information. In this case, the absorption coefficient distribution information may be acquired as photoacoustic image data. Furthermore, light with a plurality of wavelengths may be used to perform the processing in S130 and S140. Through the processing, the computing unit 151 can acquire, as photoacoustic image data, initial sound pressure distribution information or absorption coefficient distribution information corresponding to each of the plurality of wavelengths.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-220335 filed Nov. 15, 2017, which is hereby incorporated by reference herein in its entirety.

What is claimed is:

1. An information processing apparatus comprising:
an incidental information obtaining unit configured to obtain incidental information of a plurality of photoacoustic image data pieces designated based on a user's instruction in a photoacoustic image data set;
a type information obtaining unit configured to obtain type information indicating a type of composition image data; and
an adaptability acquiring unit configured to acquire, based on the incidental information, an adaptability between a combination of the plurality of photoacoustic image data pieces and computation of the composition image data of the type indicated by the type information.

2. The information processing apparatus according to claim 1, wherein the incidental information includes information indicating at least one of a measured wavelength, an image type, a photographed date and time, and patient information.

3. The information processing apparatus according to claim 2,
wherein the incidental information includes information indicating a measured wavelength, and
wherein the adaptability acquiring unit acquires the adaptability between a combination of a plurality of measured wavelengths corresponding to the plurality of photoacoustic image data pieces and computation of the composition image data based on the incidental information and the type information.

4. The information processing apparatus according to claim 1,
wherein the type information obtaining unit obtains the type information indicating a type of the composition image data designated based on a user's instruction among a plurality of types of composition image data.

5. The information processing apparatus according to claim 1, further comprising:
a composition image data computing unit configured to compute the composition image data indicated by the type information by using the plurality of photoacoustic image data pieces designated based on a user's instruction when the adaptability satisfies a predetermined condition; and
a display control unit configured to cause a display unit to display information based on the adaptability in a case where the adaptability does not satisfy the predetermined condition.

6. An information processing apparatus comprising:
a designation information obtaining unit configured to obtain designation information indicating a first photoacoustic image data designated based on a user's instruction in a photoacoustic image data set;
an incidental information obtaining unit configured to obtain incidental information of the photoacoustic image data set;
a type information obtaining unit configured to obtain type information indicating a type of composition image data; and
a display control unit configured to determine a second photoacoustic image data piece based on the incidental information, the second photoacoustic image data piece being adaptable for computing the composition image data indicated by the type information in the photoacoustic image data set and being adaptable for a combination with the first photoacoustic image data piece indicated by the designation information, the display control unit causing a display unit to display information for identifying the second photoacoustic image data piece.

7. The information processing apparatus according to claim 6, wherein the incidental information includes information indicating at least one of a measured wavelength, an image type, a photographed date and time, and patient information.

8. The information processing apparatus according to claim 7,
wherein the incidental information includes information indicating a measured wavelength, and
wherein the display control unit determines the second photoacoustic image data piece photographed with irradiation light with the measured wavelength, the measured wavelength being adaptable for computing the composition image data with respect to a measured wavelength corresponding to the first photoacoustic image data.

9. The information processing apparatus according to claim 6, wherein the type information obtaining unit obtains the type information indicating a type of the composition image data designated based on a user's instruction among a plurality of types of composition image data.

10. The information processing apparatus according to claim 6, wherein the display control unit
causes the display unit to items representing the photoacoustic image data pieces, and
determines display modes for the items such that an item representing the second photoacoustic image data piece is identified among the items, and causes the display unit to display the items in the display modes as information for identifying the second photoacoustic image data piece.

11. An information processing apparatus, comprising:
an incidental information obtaining unit configured to obtain incidental information of photoacoustic image data pieces;
a type information obtaining unit configured to obtain type information indicating a type of composition image data; and
a display control unit configured to determine a combination of photoacoustic image data pieces based on the incidental information regarding the type indicated in the type information in the photoacoustic image data set, the combination of photoacoustic image data pieces being adaptable for computing composition image data, the display control unit causing a display unit to display information indicating the combination.

12. The information processing apparatus according to claim 11, wherein the incidental information includes information indicating at least one of a measured wavelength, an image type, a photographed date and time, and patient information.

13. The information processing apparatus according to claim 12,
wherein the incidental information includes information indicating a wavelength of irradiation light, and
wherein the display control unit causes the display unit to display information indicating the combination of photoacoustic image data pieces corresponding to a combination of measured wavelengths being adaptable for computing the composition image data.

14. The information processing apparatus according to claim 11, wherein the type information obtaining unit obtains the type information indicating a type of the composition image data designated based on a user's instruction among a plurality of types of composition image data.

15. The information processing apparatus according to claim 11, wherein the display control unit
causes the display unit to items representing the photoacoustic image data pieces, and
determines display modes for the items such that a plurality of items corresponding to the combination of photoacoustic image data pieces being adaptable for computing the composition image data of the type indicated in the type information is identifiable among the items.

16. The information processing apparatus according to claim 11, wherein the display control unit determines a plurality of combinations of the photoacoustic image data pieces being adaptable for computing the composition image data of the type indicated in the type information in the photoacoustic image data set.

17. The information processing apparatus according to claim 11,
wherein the incidental information obtaining unit obtains patient information as the incidental information, and
wherein the display control unit causes the display unit to display the type of composition image data in an identifiable manner, the composition image data being computed from a combination of photoacoustic image data pieces corresponding to the patient information.

18. An information processing method comprising:
obtaining incidental information of a plurality of photoacoustic image data pieces designated based on a user's instruction in a photoacoustic image data set;
obtaining type information indicating a type of composition image data; and
to acquiring, based on the incidental information, an adaptability between a combination of the plurality of photoacoustic image data pieces and computation of the composition image data of the type indicated by the type information.

19. An information processing method comprising:
obtaining designation information indicating a first photoacoustic image data designated based on a user's instruction in a photoacoustic image data set;
obtaining incidental information of the photoacoustic image data set;
obtaining type information indicating a type of composition image data; and
determining a second photoacoustic image data piece based on the incidental information, the second photoacoustic image data piece being adaptable for computing the composition image data indicated by the type information in the photoacoustic image data set and being adaptable for a combination with the first photoacoustic image data piece indicated by the designation information, and
causing a display unit to display information for identifying the second photoacoustic image data piece.

20. An information processing method, comprising:
obtaining incidental information of photoacoustic image data pieces;
obtaining type information indicating a type of composition image data; and
determining a combination of photoacoustic image data pieces based on the incidental information regarding the type indicated in the type information in the photoacoustic image data set, the combination of photoacoustic image data pieces being adaptable for computing composition image data, and
causing a display unit to display information indicating the combination.

21. A non-transitory storage medium storing a program for causing a computer to execution the information processing method according to claim 18.

22. A non-transitory storage medium storing a program for causing a computer to execution the information processing method according to claim 19.

23. A non-transitory storage medium storing a program for causing a computer to execution the information processing method according to claim 20.
